(19) United States
(12) Patent Application Publication
(10) Pub. No.: US 2023/0338701 A1
(43) Pub. Date: Oct. 26, 2023

(54) IMPLANTABLE MEDICAL DEVICES WITH ENHANCED BIOCOMPATIBILITY

(71) Applicant: University of Connecticut, Farmington, CT (US)

(72) Inventors: Diane J. Burgess, Storrs, CT (US); Jia He, Willington, CT (US); Quanying Bao, Willimantic, CT (US)

(21) Appl. No.: 18/027,384

(22) PCT Filed: Sep. 21, 2021
(86) PCT No.: PCT/US2021/051257

§ 371 Date: Mar. 21, 2023

Related U.S. Application Data
(60) Provisional application No. 63/080,894, filed on Sep. 21, 2020.

(51) Int. Cl. A61M 25/00; A61L 29/06; A61K 31/573; A61K 38/28; A61L 29/14; A61L 29/16

(52) U.S. Cl. CPC A61M 25/005; A61L 29/06; A61K 31/573; A61K 38/28; A61M 25/0012; A61L 29/146; A61L 29/16; A61M 2025/0004; A61L 2300/43; A61L 2300/252

(57) ABSTRACT

Disclosed herein is a catheter comprising an outer conduit that comprises a first polymer; an inner conduit that comprises a second polymer; a structural support intermittently disposed in a region between the outer conduit and the inner conduit; where the structural support contacts the inner conduit and the outer conduit; where the structural support comprises a first biological agent that is released to a region outside the catheter via the outer conduit. Disclosed herein too is a method of using the catheter comprising disposing a catheter into a body of a living being; releasing the first biological agent into the organ in a first direction; and releasing a second biological agent into the organ in a second direction different from the first direction.

### CROSS REFERENCE TO RELATED APPLICATIONS

The present application claims priority to U.S. Provisional Patent Application No. 63/080,894, filed Sep. 21, 2020, which is incorporated herein in its entirety for all purposes.

### BACKGROUND

This disclosure relates to implantable medical devices with enhanced biocompatibility. In particular, this disclosure relates to an implantable catheter with enhanced biocompatibility and longevity.

The leading cause of catheter obstruction is the foreign body reaction, which leads to a fibrotic response occurring at the implantation site. Foreign body reaction is a tissue response against implanted materials. It may result in the removal of the foreign body from the body of the living being. Catheters are used for treatments such as hemodialysis, injection of insulin via an insulin pump and chemotherapy. In such applications, catheter obstruction generally occurs at a tip located at a distal end of the catheter and then the fibrotic tissue gradually covers the entire catheter.

It is therefore desirable to develop catheters that can be inserted in the body without undergoing foreign body reactions or with minimal foreign body reactions.

### SUMMARY

Disclosed herein is a catheter comprising an outer conduit that comprises a first polymer; an inner conduit that comprises a second polymer; a structural support intermittently disposed in a region between the outer conduit and the inner conduit; where the structural support contacts the inner conduit and the outer conduit; where the structural support comprises a first biological agent that is released to a region outside the catheter via the outer conduit.

Disclosed herein too is a method of using the catheter comprising disposing a catheter into a body of a living being; releasing the first biological agent into the organ in a first direction; and releasing a second biological agent into the organ in a second direction different from the first direction.

Disclosed herein too is a method of manufacturing a catheter comprising disposing on an inner conduit one or more supporting structures; where the supporting structures are reservoirs for a first biological agent; and disposing the inner conduit with the one or more supporting structures contained thereon into an outer conduit.

### BRIEF DESCRIPTION OF THE DRAWINGS

FIG. 1 is a schematic depiction of an exemplary catheter;

FIG. 2 is a graph showing the catheter performance with the same weight loading of different channeling agents in dexamethasone; and

FIG. 3 shows the ability of dexamethasone-loaded polydimethylsiloxane reservoir to reduce the fibrotic response.

### DETAILED DESCRIPTION

Disclosed herein is a medical device that comprises an outer conduit through which a biologically active agent can be provided to prevent or to minimize foreign body reactions, which result in the rejection of the medical device. In an embodiment, the provision of the biologically active agent occurs via diffusion from a supporting structure that is disposed on an inner conduit. The medical device is flexible thereby permitting easy insertion into the body of a living being. This flexibility also facilitates prevention or minimization of foreign body reactions.

In an embodiment, the medical device is a catheter that comprises a flexible outer conduit, a flexible inner conduit that is concentric with the outer conduit and an intermittent supporting structure between the outer conduit and the inner conduit. The supporting structure serves as a reservoir for a biologically active agent and supplies the biologically active agent to the exterior of the outer conduit.

The use of an intermittent supporting structure permits the catheter to be flexible. When a continuous supporting structure is placed between the inner and outer conduits it facilitates a change in the mechanical properties of the catheter, resulting in increased rigidity. Increased catheter rigidity may cause tissue irritation and further contribute to the magnitude of the foreign body response. Instead of inserting one entire piece of supporting structure between the outer and inner conduits a placement of multiple smaller intermittent supporting structures will reduce the rigidity caused by the addition of the coating.

Another aspect of the present invention is fabricating the coating with different release rates of the biologically active agent at different locations, which is beneficial for minimizing the total amount of the agent released per day and thus reducing the potential for toxicity.

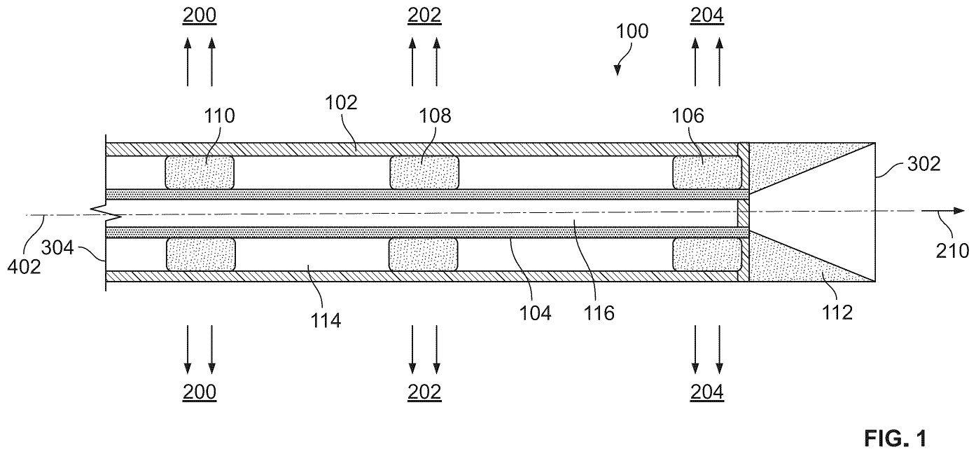

FIG. 1 discloses a catheter 100 that comprises an outer conduit 102 and an inner conduit 104 that are concentrically arranged with respect to each other and are concentrically arranged with respect to the longitudinal axis 402 of the catheter. Located intermittently between the inner conduit 104 and the outer conduit are a series of supporting structures 106, 108, 110, and so on (hereinafter just referred to as supporting structures). These supporting structures surround the inner conduit 104 and are concentrically mounted with respect to the inner conduit 104 and the outer conduit 102. The supporting structures contain a first biological agent that can be supplied to the region surrounding the catheter (when placed in an organ in the body of a living being) to prevent the foreign body response. The space between successive supporting structures 106 and 108 or between supporting structures 108 and 110 and bounded by the inner conduit 104 and outer conduit 102 is called the intermediate space 114. The intermediate space 114 is filled with a fluid. A fluid includes a liquid, a gas, or a combination thereof.

The catheter has a distal end 302 and a proximal end 304. Located at the distal end 302 is a conical shaped opening formed by a conical walled conduit 112 through which a second biological agent may be transferred to the targeted organ of the living being. While the FIG. 1 depicts a conical opening at the distal end of the catheter, other shapes such as a circular opening that is tubular may be used. In other words, the opening may have any desirable shape. The proximal end 304 is in fluid communication with a pump (not shown) that discharges the second biological agent to a targeted organ in the body of a living being. The first biological agent is released from the catheter in a different direction from the second biological agent. As will be detailed later the first biological agent is released from the catheter in a radial direction (a first direction), while the second biological agent is released from the catheter in a direction of the longitudinal axis of the catheter (a second direction). The first direction is therefore different from the second direction. While the first direction is typically different from the second direction, it can on occasion be the same. The radial direction is measured from the longitudinal axis 402 of the catheter 100 towards a circumference of the outer tube 102.

With reference now again to FIG. 1, the outer conduit 102 comprises a first polymer through which the first biological agent can be supplied to the organ of a living being. The first biological agent is contained in the supporting structures and diffuses through the polymer used in the outer conduit 102 to contact the organ that the catheter 100 is in contact in. The first polymer should be biocompatible and should preferably not react with the biological agents that it delivers to the organ in which it is disposed. The first polymer should be flexible so that the catheter can flex (so that it can travel a tortuous path during insertion into the organ) and should be capable of being temporarily deformed so that it can return to its original shape after the deforming force is removed. The polymer should also allow diffusion of the biological agent through it, while preventing diffusion of bodily fluids into the catheter. To display these properties, the first polymer is preferably an elastomer or if not, an elastomer is in the form of a film that is thin enough to flex without undergoing deformation. The first polymer is preferably an amorphous polymer. In an embodiment, the first polymer may also be crosslinked.

The first polymer is an organic polymer that may be selected from a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can be an oligomer, a homopolymer, a copolymer, a block copolymer, an alternating block copolymer, a random polymer, a random copolymer, a random block copolymer, a graft copolymer, a star block copolymer, a dendrimer, an ionomer, or the like, or a combination thereof. The organic polymers have number average molecular weights greater than 10,000 grams per mole, preferably greater than 20,000 g/mole and more preferably greater than 50,000 g/mole.

Organic polymers that may be used in the outer conduit or in the inner conduit include polyacetals, polyacrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyimides, polyetherimides, polytetrafluoroethylenes, polyetherketones, polyether ether ketones, polyether ketone ketones, polybenzoxazoles, polyoxadiazoles, polybenzothiazinophenothiazines, polybenzothiazoles, polypyrazinoquinoxalines, polypyromellitimides, polyguinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, poly dibenzofurans, polyphthalides, polyacetals, poly anhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, poly sulfonamides, polyureas, polyphosphazenes, polysilazanes, polypropylenes, polyethylenes, polyethylene terephthalates, polyvinylidene fluorides, polysiloxanes, or the like, or a combination thereof.

In an embodiment, the outer conduit comprises a first polymer that is an elastomer. The first elastomer is preferably biocompatible. Examples of elastomers that may be used in the outer conduit include polybutadienes, polyisoprenes, styrene-butadiene rubber, poly(styrene)-block-poly(butadiene), poly(acrylonitrile)-block-poly(styrene)-block-poly(butadiene) (ABS), polychloroprenes, epichlorohydrin rubber, polyacrylic rubber, silicone elastomers (polysiloxane), fluorosilicone elastomers, fluoroelastomers, perfluoroelastomers, polyether block amides (PEBA), chlorosulfonated polyethylene, ethylene propylene diene rubber (EPR), ethylene-vinyl acetate elastomers, or the like, or a combination thereof. In a preferred embodiment, the first polymer (used on the outer conduit 102) is elastomeric and comprises a polysiloxane, a fluorosilicone elastomer, a fluoroelastomer or a perfluoroelastomer. In another embodiment, the first polymer is a polysiloxane.

The inner conduit 104 comprises a second polymer. The second polymer may be the same or different from the first polymer and is also biocompatible. In an embodiment, the second polymer is different from the first polymer. The second polymer is preferably stiffer than the first polymer and has a higher modulus of elasticity than the first polymer. The second polymer is also preferably flexible and prevents diffusion of the first biologically active agent into the interior passage 116 of the inner conduit 104. The second polymer prevents diffusion of the second biologically active agent (that flows through the inner conduit 104) from contacting the first biologically active agent.

The second polymer can comprise one or more of the polymers listed above. The second polymer preferably comprises a semi-crystalline polymer. The second polymer is preferably a barrier layer that prevents the first biological agent from diffusing into the channel 116. In a preferred embodiment, the second polymer comprises a polyolefin. Examples of suitable polyolefins are polyethylene, polypropylene, copolymers of polyethylene or polypropylene with other α-olefins. α-olefins having 3 to 12 carbon atoms are preferred.

Suitable examples of polyolefins for use in the inner conduit 104 are ultralow density polyethylene (ULDPE), low density polyethylene (LDPE), linear low density polyethylene (LLDPE), medium density polyethylene (MDPE), high density polyethylene (HDPE), high melt strength high density polyethylene (HMS-HDPE), ultrahigh density polyethylene (UHDPE), or a combination thereof. In a preferred embodiment, the second polymer is low density polyethylene or high density polyethylene that is of a thickness that renders the catheter flexible while at the same time preventing diffusion of the first biological agent with the second biological agent contained in the inner conduit.

Disposed intermittently in the intermediate space between the inner conduit 104 and the outer conduit 102 are a plurality of concentrically mounted supporting structures 106, 108, 110, and so on. The supporting structures are for example, donut shaped with an inner surface that preferably contacts an outer surface of the inner conduit 104 (preferably along the entire outer circumference) and an outer surface that contacts an inner surface of the outer conduit 102 (preferably along the entire inner circumference). The donut shape is an exemplary shape but any other shape that supports the outer conduit may be used. Similarly, the supporting structure does not have to contact the outer surface of the inner tube. In other words, the shape and structure of the supporting structure is selected to facilitate transport of the biological agent while at the same time not substantially increasing the stiffness (e.g., the elastic modulus) of the catheter. In one such embodiment, the supporting structure does not continuously contact the outer surface of the inner tube and does not continuously contact an inner surface of the outer tube.

The supporting structures also serve as reservoirs for the first biological agent which minimizes or eliminates foreign body reactions. In an embodiment, the plurality of supporting structures contain the first biological agent along with an optional channeling agent. The channeling agent facilitates release of the first biological agent from the reservoir through the outer conduit to the surrounding tissue (of the organ into which the catheter is inserted). The space 114 between the supporting structures is filled with a fluid such as, for example air. Other fluids (e.g., water, oil, inert gases, and the like) may also be used if desired.

The supporting structures are preferably manufactured from a material that can retain the first biological agent without expelling it over time (because of compatibility issues) while at the same time providing support for the outer conduit without appreciably increasing the stiffness or weight of the catheter. In an embodiment, the supporting structures are manufactured from a material that is compatible with the first biological agent over a period of time and over a range of different temperatures that vary from −10° C. to 50° C., preferably +10° C. to 30° C.

The supporting structures are spaced at a distance apart to prevent increasing the stiffness of the catheter and are present (in the catheter) in a number effective to supply the first biological agent to tissue surrounding the catheter (when it is disposed in an organ in the body of a living being). The minimum distance between successive supporting structures is selected to prevent any increases in flexibility of the catheter. In an embodiment, the successive structures are periodically spaced along the longitudinal axis 402 of the catheter 100. In another embodiment, the supporting structures are aperiodically spaced along the longitudinal axis 402 of the catheter. In summary, the plurality of structural supports that are spaced so as not to reduce a flexibility of the catheter when compared with an equivalent catheter that does not contain the plurality of structural supports.

In an embodiment, the flexural modulus of the catheter with the structural supports is not increased by an amount of greater than 5%, preferably not greater than 10%, and more preferably not greater than 15%, when compared with another catheter that has a similar structure without the structural supports, or alternatively, with the structural supports being closer to each other.

The supporting structures may be designed so that the release rate of the first biological agent may be different for different supporting structures. This is discussed below.

In an embodiment, the supporting structure comprises a third polymer that may be the same or different from the first polymer and the second polymer. The third polymer may be selected from the list of polymers mentioned above.

In an embodiment, the third polymer is different from the first polymer as well as from the second polymer. In yet another embodiment, the third polymer is preferably the same as the first polymer and comprises a polysiloxane, a fluorosilicone elastomer, a fluoroelastomer or a perfluoroelastomer. In a preferred embodiment, the third polymer used in the supporting structures is a polysiloxane.

In an embodiment, the supporting structure at different locations in the catheter may have different structures so that the release rate of the first biological agent may be varied depending upon its location in the catheter. For example, with reference to the FIG. 1, the supporting structure 106 has a higher release rate (of the first biological agent) than the next supporting structure 108. Similarly, the supporting structure 108 may have a higher release rate than the supporting structure 110. The release rate of the different structures in the catheter may be adjusted depending upon their location in the organ. In one embodiment, the release rate for the plurality of supporting structures may be sequentially varied. In another embodiment, the release rate for each supporting structure is varied randomly depending upon the location of the supporting structure in the catheter and the catheter's location in the organ.

In one embodiment, the supporting structure comprises a polymeric foam. The foam can store the first biological agent and release it as needed. The release of the first biological agent occurs outwards in the radial direction (a first direction) from the longitudinal axis 402. The direction of release is reflected by arrows 200, 202, 204, and so on in the FIG. 1. By varying the pore size of the foam used at different locations, the discharge rate of the biological agent may be varied. Since capillarity pressure varies inversely with pore radius, the pore size of the foam can be varied to vary discharge rates of the biological agent.

In another embodiment, the discharge rate of the biological agent may be varied by using a channeling agent in conjunction with the biological agent. Channeling agents are described later.

The first biological agent and the second biological agent are different in chemical composition from each other but may contain some common ingredients. As may be seen in the FIG. 1, the first biological agent diffuses radially outwards from the supporting structure through the first polymer (used in the outer conduit 102) (the first direction), while the second biological agent is transported through passage 116 in the inner conduit 104 and is released in the direction of the longitudinal axis (the second direction as indicated by the arrow 210). The second biological agent is pumped from the proximal end 304 to the distal end 302 and is delivered to the surrounding tissue via the conical opening in the conical conduit 112.

The first biological agent and the second biological agent may be therapeutically and pharmaceutically biologically active agents that include anti-proliferative/antimitotic agents including natural products such as vinca alkaloids (e.g., vinblastine, vincristine, and vinorelbine), paclitaxel, epidipodophyllotoxins (e.g., etoposide, teniposide), antibiotics (e.g., dactinomycin, actinomycin D, daunorubicin, doxorubicin, penicillin V, penicillin G, ampicillin, amoxicillin, cephalosporin, tetracycline, doxycycline, minocycline, demeclocycline, erythromycin, aminoglycoside antibiotics, polypeptide antibiotics, nystatin, griseofulvin, and idarubicin), anthracyclines, mitoxantrone, bleomycins, plicamycin, mithramycin and mitomycin, enzymes (L-asparaginase, which systemically metabolizes L-asparagine and deprives cells which do not have the capacity to synthesize their own asparagine), antiplatelet agents such as G(GP) IIb/IIIa inhibitors and vitronectin receptor antagonists, anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), anti-proliferative/antimitotic antimetabolites such as folic acid analogs (e.g., methotrexate), pyrimidine analogs (e.g., fluorouracil, floxuridine, cytarabine), purine analogs and related inhibitors (e.g., mercaptopurine, thioguanine, pentostatin and 2-chlorodeoxyadenosine {cladribine}), platinum coordination complexes (e.g., cisplatin, carboplatin), procarbazine, hydroxyurea, mitotane, aminoglutethimide, hormones (e.g., estrogen), anti-coagulants (e.g., heparin, synthetic heparin salts and other inhibitors of thrombin), fibrinolytic agents (e.g., tissue plasminogen activator, streptokinase and urokinase), aspirin, dipyridamole, ticlopidine, clopidogrel, abciximab, antimigratory, antisecretory (e.g., breveldin), anti-inflammatory: such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives such as aspirin, para-aminophenol derivatives such as acetominophen, indole and indene acetic acids (e.g., indomethacin, sulindac, etodalac), heteroaryl acetic acids (e.g., tolmetin, diclofenac, ketorolac), arylpropionic acids (e.g., ibuprofen and derivatives), anthranilic acids (e.g., mefenamic acid, meclofenamic acid), enolic acids (e.g., piroxicam, tenoxicam, phenylbutazone, oxyphenthatrazone), nabumetone, gold compounds (e.g., auranofin, aurothioglucose, gold sodium thiomalate), immunosuppressives (e.g., cyclosporine, tacrolimus (FK-506), sirolimus (e.g., rapamycin, azathioprine, mycophenolate mofetil), angiogenic agents such as vascular endothelial growth factor (VEGF), fibroblast growth factor (FGF), angiotensin receptor blockers, nitric oxide donors, anti-sense oligionucleotides and combinations thereof, cell cycle inhibitors, mTOR inhibitors, and growth factor receptor signal transduction kinase inhibitors, retenoids, cyclin/CDK inhibitors, HMG co-enzyme reductase inhibitors (statins), protease inhibitors, anabolic inhibitors (e.g., insulin), or the like, or a combination thereof.

In one exemplary embodiment, the second biological agent comprises insulin, dialysate, chemotherapeutics, non-steroidal agents, steroids, angiogenic agents, or the like, or a combination thereof.

In a preferred embodiment, the first biologically active agent is not the same as the second biologically active agent. In a preferred embodiment, the first biologically active agent is dexamethasone (which is delivered through the outer conduit 102) while the second biologically active agent is insulin (which is delivered through channel 116 in the inner conduit 104).

As noted above, the plurality of supporting structures contain the first biological agent in addition to a channeling agent. The channeling agent facilitates release of the first biological agent from the supporting structure through the outer conduit to the surrounding tissue. The channeling agent is preferably biocompatible and acts an osmotic agent. It enables control of the release rate of the biologically active agent. It may sometimes adjust the viscosity of biologically active agent solutions by altering the ionic attributes of a formulation.

Examples of channeling agents are salts, polyalkylene glycols, naturally occurring polymers, biodegradable polymers, or the like, or a combination thereof.

Examples of salts (organic and inorganic salts) for use as channeling agents include sodium chloride, potassium chloride, bicarbonate salts, magnesium salts, phosphate and sulfate salts, or the like, or a combination thereof.

Examples of polyalkylene glycols for use as channeling agents include polyethylene glycol, poly propylene glycol, polytetramethylene oxide, or the like, or a combination thereof.

The polyalkylene glycols have a weight average molecular weight of 400 to 15,000 grams per mole, preferably 1200 to 10,000 grams per mole.

Examples of naturally occurring polymers for use as channeling agents include cellulose and cellulose derivatives (e.g., hydroxymethylcellulose, hydroxyethylcellulose, hydroxypropylcellulose, hydroxypropyl methylcellulose, sodium carboxymethyl cellulose, cellulose acetate, cellulose diacetate, cellulose triacetate, cellulose propionate, cellulose acetate butyrate, and cellulose ethers like ethyl cellulose, or the like, or a combination thereof), sugars (glucose, sucrose, lactose, galactose, fructose, mannitol, sorbitol, or a combination thereof), ionic complexes of celluloses, gums (e.g., acacia, alginate, carrageenan, guar, karaya, pectin, tragacanth, xanthan, or the like, or a combination thereof), or a combination thereof.

Suitable examples of biodegradable polymers are as polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or the like, or a combination thereof.

Non-biodegradable polymers such as EUDRAGIT (which is the brand name for a diverse range of polymethacrylate-based copolymers) that includes anionic, cationic, and neutral copolymers based on methacrylic acid and methacrylic/acrylic esters or their derivatives) can also work as a channeling agent. In other words, biodegradable and non-biodegradable polymers can be used as channeling agents.

The channeling agent may be added in amounts of 1% to 50% weight percent based on the total weight of the channeling agent and the first biological agent.

In one embodiment, in one method of manufacturing the catheter 100, the inner conduit 104 is fitted with the supporting structures 106, 108, 110 (previously saturated with the biologically active agent and optional channeling agent) and the combination is then disposed into the outer conduit 102. The catheter 100 is then fitted with the conical conduit 112 at the distal end 302 and packaged for use.

In using the catheter 100 disclosed herein, the proximal end 304 of the catheter 100 is connected to a pump (not shown). The pump is in fluid communication with a reservoir that contains the second biological agent (e.g., insulin). The catheter 100 is then implanted into the body of the living being. After insertion, insulin is discharged via the conical conduit 112 located at the distal end of the tube. During the existence of the catheter in the organ, the first biological agent (e.g., dexamethasone) will be released from the supporting structures 106, 108, 110, and so on, through the outer conduit 102 into the tissue surrounding the catheter 100 to prevent or to minimize a foreign body reaction. The release of the first biological agent to the surrounding tissue can take place over extended periods of time, thus facilitating an extended life cycle for the catheter described herein.

The functioning of the catheter detailed herein is described in the following non-limiting example.

### EXAMPLE

This example was conducted to demonstrate the functioning of the implantable catheter disclosed herein. The outer conduit comprises polydimethylsiloxane, while the inner conduit comprises polyethylene. All supporting structures contain dexamethasone with a channeling agent. The polymer used in the supporting structure is polydimethylsiloxane. A single type of channeling agent was included with dexamethasone in each supporting structure.

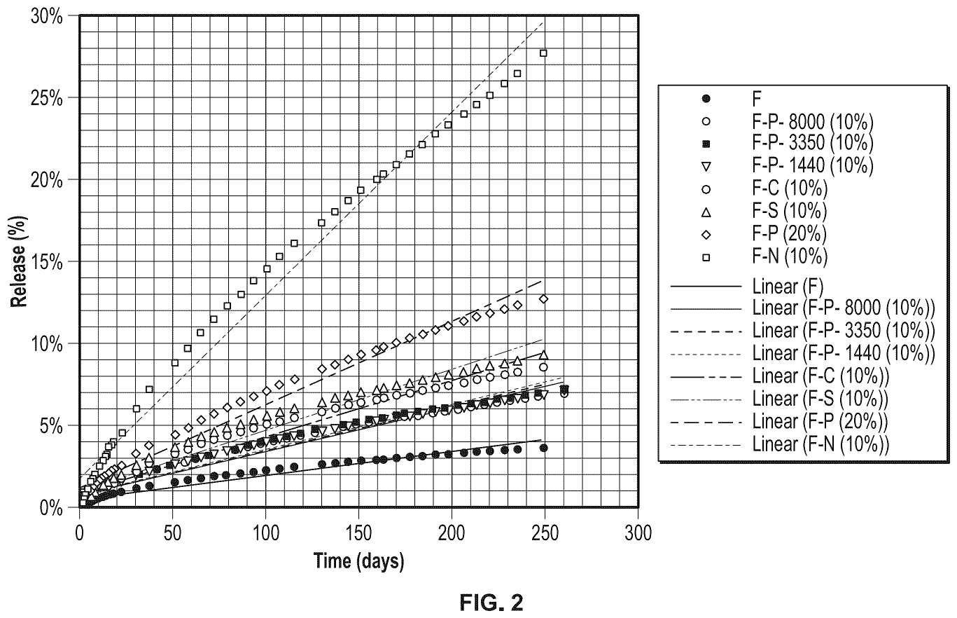

FIG. 2 is a graph showing the in vitro release pattern of a dexamethasone-loaded polydimethylsiloxane supporting structure (reservoir) in pH 7.4 PBS phosphate-buffered saline at 37° C. All the drug reservoirs contain the same amount of dexamethasone, but different types or amounts of channeling agents, including sodium chloride, polyethylene glycol, sucrose or sodium carboxymethyl cellulose. A single type of channeling agent was incorporated in each reservoir. The polydimethylsiloxane polymeric system showed a controlled capability of drug release over a long duration. In the absence of a channeling agent, approximately 3.6% of the dexamethasone was released after 250 days without any dumping effect, showing a kinetic release rate that is near-zero order (i.e., the release follows near-zero order kinetics). The following nomenclature shown in the Table below may be used to interpret FIG. 2.

TABLE

| Legend | Interpretation |
|---|---|
| F | no channeling agent |
| F-P-8000 (10%) | 10 wt % PEG with weight average |
| | molecular weight (MW) 8000 g/mole |
| F-P-3350 (10%) | 10 wt % PEG with MW 3350 g/mole |
| F-P-1440 (10%) | 10 wt % PEG with MW 1440 g/mole |
| F-C (10%) | 10 wt % carboxymethyl cellulose |
| F-S (10%) | 10% sucrose |
| F-P (20%) | 20 wt % PEG with MW 1440 g/mole |
| F-N (10%) | 10 wt % sodium chloride |

From the FIG. 2, it may be seen that the addition of channeling agent to the polydimethylsiloxane reservoir matrix increased the drug release rate as well as swelling ratio in the following rank order: sodium chloride>sucrose>sodium carboxymethyl cellulose>polyethylene glycol. In addition, the increased rate and extent of drug release was observed with a higher amount of channeling agent.

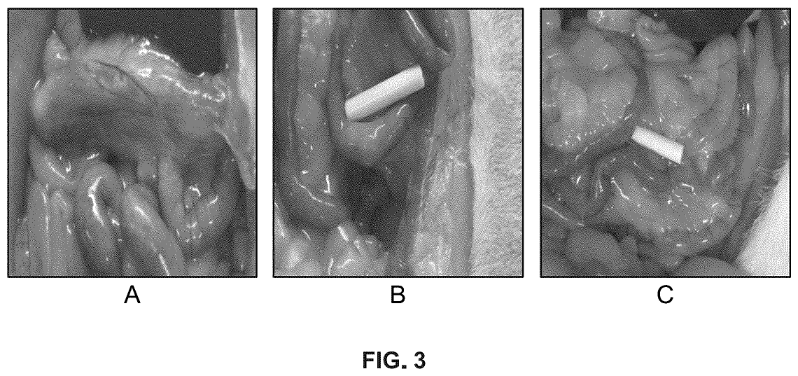

FIG. 3 shows the ability of dexamethasone-loaded polydimethylsiloxane reservoir to reduce the fibrotic response. Photograph A) displays the development of fibrotic encapsulation surrounding the intraperitoneal catheter 2 months post implantation in a rodent model. Photographs B) and C) show the situation of dexamethasone-loaded polydimethylsiloxane reservoirs at 228 days and 365 days following implantation, respectively. No fibrotic encapsulation was observed surrounding the dexamethasone-loaded polydimethylsiloxane reservoirs.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. A catheter comprising:
an outer conduit that comprises a first polymer;
an inner conduit that comprises a second polymer;
a structural support intermittently disposed in a region between the outer conduit and the inner conduit; where the structural support contacts the inner conduit and the outer conduit; where the structural support comprises a first biological agent that is released to a region outside the catheter via the outer conduit.

2. The catheter of claim 1, where the first polymer is different from the second polymer.

3. The catheter of claim 2, where the first polymer is an amorphous elastomer and where the second polymer is a semi-crystalline polymer.

4. The catheter of claim 3, where the first polymer is polydimethylsiloxane and where the second polymer is a polyolefin.

5. The catheter of claim 1, where the inner conduit transports a second biological agent from a pump to a body of a living being.

6. The catheter of claim 1, further comprising a conical conduit in contact with the outer conduit and the inner conduit at a distal end of the catheter; where the conical conduit is operative to discharge a second biological agent into a body of a living being.

7. The catheter of claim 1, wherein the first biological agent is dexamethasone.

8. The catheter of claim 5, wherein the second biological agent is insulin.

9. The catheter of claim 1, where the first biological agent is blended with a channeling agent.

10. The catheter of claim 1, where the structural support comprises the same polymer as the outer conduit.

11. The catheter of claim 1, where the structural support comprises a plurality of structural supports that are spaced so as to not substantially change a flexibility of the catheter when compared with an equivalent catheter that does not contain the plurality of structural supports.

12. The catheter of claim 11, where successive structural supports of the plurality of structural supports are periodically spaced.

13. The catheter of claim 1, where the structural support is a foam.

14. The catheter of claim 11, where at least one structural support releases the first biological agent at a different rate from another structural support.

15. The catheter of claim 1, where successive structural supports of the plurality of structural supports are aperiodically spaced.

16. A method of using the catheter of claim 1, comprising:
disposing a catheter into a body of a living being;
releasing the first biological agent into the organ in a first direction; and
releasing a second biological agent into the body of the living being in a second direction different from the first direction.

17. The method of claim 16, wherein the first direction is a radial direction and wherein the second direction is along a longitudinal axis of the catheter; wherein the radial direction is from the longitudinal axis towards a circumference of the outer tube.

18. The method of claim 16, wherein the first biological agent is dexamethasone and wherein the second biological agent is insulin.

19. A method of manufacturing a catheter comprising:
disposing on an inner conduit one or more supporting structures; where the supporting structures are reservoirs for a first biological agent; and
disposing the inner conduit with the one or more supporting structures contained thereon into an outer conduit.

20. The method of claim 19, wherein the supporting structures are spaced at a distance effective to prevent a substantial increase in stiffness of the catheter.
